(19) United States
(12) Patent Application Publication
(10) Pub. No.: US 2014/0323414 A1
(43) Pub. Date: Oct. 30, 2014

(54) INOTROPIC COMPOUNDS

(71) Applicant: YISSUM RESEARCH DEVELOPMENT COMPANY OF THE UNIVERSITY OF JERUSALEM LTD., Jerusalem (IL)

(72) Inventors: Tzachi Tal, Kiryat Ata (IL); Eli Shapira, Hadassah (IL); Nir Nesher, Kfar Ben Nun (IL); Michal Horowitz, Jerusalem (IL)

(21) Appl. No.: 14/361,370

(22) PCT Filed: Nov. 29, 2012
(86) PCT No.: PCT/IB2012/056824

§ 371 Date: May 29, 2014

Related U.S. Application Data
(60) Provisional application No. 61/565,658, filed on Dec. 1, 2011.

(51) Int. Cl. C07K 14/435; C07K 7/08

(52) U.S. Cl. CPC C07K 14/43504; C07K 7/08
USPC 514/21.3; 514/21.5

(57) ABSTRACT

The present invention provides the use of a compound which activates at least one of the sodium channels: NAV 1.1, NAV 1.3, NAV 1.6 or a combination of two or more of such sodium channels, in the preparation of a medicament for increasing cardiac output. Moreover, the aim of the invention, is utilizing this compound for increasing cardiac output without affecting cardiac rhythm or without inducing cardiac rhythm disturbance.

### FIELD OF INVENTION

A composition comprising a compound capable of specifically and differentially activating NAV sodium channels and methods for treating cardiac failure with same are provided.

### BACKGROUND OF THE INVENTION

Heart failure is characterized by the heart's inability to effectively pump blood to meet the body's demands. Some heart diseases, even in the early stages, can impair a heart cell's ability to contract and relax, which may result from the significant difference in contractile force between normal and heart failure cells. Thus, the information on contractile force of heart cells will be very helpful for understanding molecular alterations in diseased heart cells. In particular, understanding disease-induced alterations in contractile properties (such as contractile force and Young's modulus) at single cardiac myocyte level may lay the foundation for quantitatively understanding the mechanism of heart failure.

In the United States approximately 5.7 million patients suffer from heart failure (HF), and each year approximately 670000 new patients develop the condition. Its incidence approaches 10 per 1000 population after 65 years of age. The disorder is the underlying reason for 12 to 15 million office visits and 6.5 million hospital days each year. During the last 10 years, Nearly 300,000 patients die of HF as a primary or contributory cause each year, and the number of deaths has increased steadily despite advances in treatment. The estimated direct and indirect cost of HF in the United States for 2009 is $37.2 billion.

Over the last two decades progress has been achieved in the development of drugs which decrease the mechanical load on the myocardium. However, there was no great progress in developing new positive ionotropic agents which can improve the symptoms of the disease. Current treatments of heart failure include bed rest, water and salt restriction, low supplemental oxygen therapy, diuretics, vasodilators (e.g. hydralazine and nitrates), ACE inhibitors (e.g. captopril and enalapril), angiotensin II-receptor blockers, beta-adrenergic receptor blockers, aldosteron and digitalis. The most commonly used positive ionotropic drugs that used in standard therapy are cardiac glycosides, the use of which involves risk to the patients due their toxicity and narrow therapeutic window.

Digoxin, for example, has a low therapeutic index (0.5 to 0.8 ng/mL) and is associated with a high risk of toxicity, including arrhythmias. There is a great necessity to developing new inotropic drugs.

Pompilidotoxins (PMTXs) are derived from venom and are known to facilitate synaptic transmission and slows Na+ channel inactivation without modifying activation process. β-PMTX has been purified from the venom of the spider wasp and identified as a novel polypeptide neurotoxin with 13 amino acid residues and molecular mass of approximately 1530 Da (Konno et al., 1998; Miyawaki et al., 2002). β-PMTX modifies rat brain type II Na+ channel α-subunit (rBII) expressed in human embryonic kidney cells but fails to act on the rat heart α-subunit (rH1) at similar concentrations. β-PMTX causes a slowing of inactivation process in TTX-sensitive Na+ channels from rat trigeminal neurons. α-PMTX in which lysine at position 12 of β-PMTX is replaced by arginine, was found to induce a facilitation of both excitatory and inhibitory postsynaptic potentials, suggesting a possibility of increase in the firing frequency of Na+ channels (Konno et al., 1997, 1998; Harsch et al., 1998).

Because voltage-dependent Na+ channels are a main component for the generation of the rapid depolarization during the initial phase of action potential, many natural toxins are designed to modify their functions so to capture a prey and to defend itself from predators. These Na+ channel-specific natural toxins are very useful tools for understanding and correlating the structure and function of Na+ channel (Catterall, 1980, 1995, 2000; Strichartz et al., 1987).

Voltage-gated sodium channels composed of pore-forming α and auxiliary β subunits are responsible for the rising phase of the action potential in cardiac muscle, but the functional roles of distinct sodium channel subtypes have not been defined. Immunocytochemical studies show that the principal cardiac performing a subunit isoform Nav1.5 is preferentially localized in intercalated disks, whereas the brain a subunit isoforms Nav1.1, Nav1.3, and Nav1.6 are localized in the transverse tubules. Sodium currents due to the highly tetrodotoxin (TTX)-sensitive brain isoforms in the transverse tubules are small and are detectable only after activation with β scorpion toxin (Maier S K G. et al. 2002).

### SUMMARY OF THE INVENTION

The present invention provides, in one embodiment, the use of a compound which activates at least one of the sodium channels: NAV 1.1, NAV 1.3, NAV 1.6 or a combination of two or more of such sodium channels, in the preparation of a medicament for increasing cardiac output. The compound, in another embodiment, does not activate the sodium channel NAV 1.5. In some embodiments, the compound activates sodium channel NAV 1.5 to a lesser extent compared to its activation of sodium channel NAV 1.1, sodium channel NAV 1.3, and sodium channel NAV 1.6.

The compound of the invention, in some embodiments, is a peptide, a polypeptide or a protein. In some embodiments, the compound is a peptide comprising the amino acid sequence of beta-PMTX, a fragment or a mimetic having the biological activity as described above (differential activation of sodium channels). In some embodiments, the compound is a peptide comprising the amino acid sequence of delta-EVIA, a fragment or a mimetic having the biological activity as described above (differential activation of sodium channels). In some embodiments, the compound is a peptide comprising the amino acid sequence of beta-scorpion toxin (Css IV), a fragment or a mimetic having the biological activity as described above (differential activation of sodium channels).

The aim of the invention, in some embodiments, is utilizing a compound as described herein for increasing cardiac output. Increasing cardiac output is achieved, according to some embodiments, without affecting cardiac rhythm or without inducing cardiac rhythm disturbance. Increasing cardiac output, in another embodiment, is free or substantially free of affecting an electrical property or conductance of the cardiac tissue. Increasing cardiac output, in some embodiments, is increasing the amplitude of a cardiomyocyte contraction. In another embodiment, increasing cardiac output is increasing the force or energy of cardiac contraction. In another embodiment, increasing cardiac output is increasing left ventricular pressure.

In some embodiments, the invention further provides that increasing cardiac output is inhibiting or ameliorating a symptom associated with heart failure. Symptoms associated with heart failure include but are not limited to: edema, ascites, nocturia, hepatomegaly, jaundice, coagulopathy, dyspnea, orthopnea, fatigability, cardiac asthma, or any combination thereof.

### BRIEF DESCRIPTION OF THE DRAWINGS

FIG. 1. A graph showing the effect of δ-EVIA on heart pressure output. As shown, the treatment does not affect heart rate.

FIG. 2. A bar graph showing that δ-EVIA affect heart twitch kinetic in a concentration dependent manner. δ-EVIA clearly increased the heart ability of producing pressure at a given time (top graph) (positive ΔP/ΔT). B) δ-EVIA clearly increased the heart ability to regain relaxation (bottom graph) (negative ΔP/ΔT).

FIG. 3. A graph showing the effects of PMTX on cardiomyocytes contractions. A) Twitches series before and after applications of PMTX 10 μM. B) Average of 14 twitches before and after the applications of PMTX 10 μM. As can be seen, the toxin increased significantly the twitches amplitude with no effects on twitches configuration and durations. C) PMTX affected cardiomyocytes contractions in concentration dependent manner.

FIG. 4. A bar graph showing that β-PMTX increases ex-vivo heart pressure output with no significant effect on its beat rate. Values present as percentage of Control. Control represent the period of time after steady state achievement and before the application of β-PMTX.

FIG. 5. A bar graph showing that β-PMTX affect heart twitch kinetic in a concentration dependent manner. β-PMTX clearly increases the heart ability of producing pressure at a given time (positive ΔP/ΔT) and the heart ability to regain relaxation (negative ΔP/ΔT).

FIG. 6. A graph showing that β-PMTX increases LvP and the arterial blood pressure in-vivo in anesthetized rat. A) Control: LvP, ABP and ECG before the application of β-PMTX. B) LvP, ABP and ECG after the application of 1 mg β-PMTX (blood volume, 7-8 ml). C) LvP, ABP and ECG after the application of 0.1 mg adrenalin. The arrhythmogenic effect of adrenalin is clearly demonstrated.

### DETAILED DESCRIPTION OF THE INVENTION

The present invention is directed, in some embodiments, to a method for increasing cardiac output in a subject in need thereof, comprising the step of administering to the subject an effective amount of a composition comprising a compound which activates at least one of the sodium channels: NAV 1.1, NAV 1.3, NAV 1.6 or a combination of two or more of such sodium channels. Increase in cardiac output is a result in increase of stroke volume per beat, in the number of beats per minute (heart rate), or both. In another embodiment, increase in cardiac output according to the methods described herein is increase of stroke volume per beat without substantially affecting the heart rate. In another embodiment, increase in cardiac output according to the methods described herein is increase of stroke volume per beat without an induction of arrhythmia. In another embodiment, increase in cardiac output according to the methods described herein is increase in the force or energy of cardiac muscular contractions due to contraction of the cardiomyocytes. In another embodiment, increase in cardiac output according to the methods described herein is induced without significant influence on the cardiomyocytes action potential.

The present invention provides, unexpectedly, a method based on a compound of the invention, for increasing cardiac output without affecting the configuration of myocytes action potential. The unexpected advantage of the present invention includes the use of a compound that increases cardiac output without the devastating effects of cardiac rhythm disturbances. In some embodiments, the compound of the invention is a modulator that increases stroke volume per beat without significantly affecting heart rate.

In some embodiments, the invention provides a method for increasing cardiac output in a subject in need thereof, comprising administering to the subject an effective amount of a composition comprising a compound of the invention such as but not limited to: a peptide comprising the amino acid sequence of SEQ ID NO: 1, SEQ ID NO: 2, or SEQ ID NO: 3.

The methods of the invention, in some embodiments, provide that administering an effective amount of a compound of the invention to a subject in need thereof; increase the amplitude of a cardiomyocyte contraction. The methods of the invention, in some embodiments, provide that administering an effective amount of a compound of the invention to a subject in need thereof; increase the force or energy of cardiac contraction. The methods of the invention, in some embodiments, provide that administering an effective amount of a compound of the invention to a subject in need thereof; increase left ventricular pressure. The methods of the invention, in some embodiments, provide that administering an effective amount of a compound of the invention to a subject in need thereof; does not induce a cardiac rhythm disturbance. The methods of the invention, in some embodiments, provide that administering an effective amount of a compound of the invention to a subject in need thereof; is substantially free of affecting an electrical property of the cardiac muscle.

In another embodiment, increase of stroke volume per beat is increase in preload, afterload, contractility, or any combination thereof. In another embodiment, increase of stroke volume per beat is increase in the force that the heart muscle creates at the given length.

In another embodiment, the compound of the invention increases stroke volume per beat by at least 10% without substantially affecting the heart rate. In another embodiment, the compound of the invention increases stroke volume per beat by at least 20% without substantially affecting the heart rate. In another embodiment, the compound of the invention increases stroke volume per beat by at least 30% without substantially affecting the heart rate. In another embodiment, the compound of the invention increases stroke volume per beat by at least 40% without substantially affecting the heart rate. In another embodiment, the compound of the invention increases stroke volume per beat by at least 50% without substantially affecting the heart rate. In another embodiment, the compound of the invention induces positive inotropic effect in cardiomyocytes.

In another embodiment, the compound of the invention increases cardiac output to about 5 L/min. In another embodiment, the compound of the invention increases cardiac output to about 4 to 6 L/min. In another embodiment, the compound of the invention increases cardiac output to about 4.5 to 5.5 L/min. In another embodiment, the compound of the invention increases cardiac output to about 4.6 to 5.4 L/min. In another embodiment, the compound of the invention increases cardiac output to about 4.8 to 5.2 L/min.

In another embodiment, the compound of the invention increases cardiac output by at least 10%. In another embodiment, the compound of the invention increases cardiac output by at least 12%. In another embodiment, the compound of the invention increases cardiac output by at least 15%. In another embodiment, the compound of the invention increases cardiac output by at least 20%. In another embodiment, the compound of the invention increases cardiac output by at least 25%. In another embodiment, the compound of the invention increases cardiac output by at least 30%. In another embodiment, the compound of the invention increases cardiac output by at least 40%. In another embodiment, the compound of the invention increases cardiac output by at least 50%. In another embodiment, the compound of the invention increases cardiac output by at least 60%. In another embodiment, the compound of the invention increases cardiac output by at least 70%. In another embodiment, the compound of the invention increases cardiac output by at least 100%.

The phrase “without substantially affecting the heart rate” includes increase of heart rate of up to 15%, 10%, 7%, 5%, or 3%.

The compound of the invention which increases or induces the increase of stroke volume per beat, according to some embodiments, does not activate the sodium channel NAV 1.5. According to another embodiment, the compound activates sodium channel NAV 1.5 to a lesser extent compared to the compound's activation capacity of sodium channel NAV 1.1, sodium channel NAV 1.3, and sodium channel NAV 1.6. In another embodiment, activation to a lesser extent of a sodium channel means that only high dosages that are beyond the therapeutic window (the dosage range) of the methods described herein can activate sodium channel NAV 1.5. In another embodiment, activation to a lesser extent means that activation of the sodium channel NAV 1.5 results in a reduced current compared to activation of sodium channel NAV 1.1, sodium channel NAV 1.3, and sodium channel NAV 1.6 by a given dosage (within the dosage range of the invention) of the same compound. In another embodiment, activation to a lesser extent of NAV 1.5 is at least twice less activation of NAV 1.5 compared to the activation of: sodium channel NAV 1.1, sodium channel NAV 1.3, and sodium channel NAV 1.6, with a compound at a given dosage. In another embodiment, activation to a lesser extent of NAV 1.5 is at least 3 times less activation of NAV 1.5 compared to the activation of: sodium channel NAV 1.1, sodium channel NAV 1.3, and sodium channel NAV 1.6, with a compound at a given dosage. In another embodiment, activation to a lesser extent of NAV 1.5 is at least 4 times less activation of NAV 1.5 compared to the activation of: sodium channel NAV 1.1, sodium channel NAV 1.3, and sodium channel NAV 1.6, with a compound at a given dosage. In another embodiment, activation to a lesser extent of NAV 1.5 is at least 5 times less activation of NAV 1.5 compared to the activation of: sodium channel NAV 1.1, sodium channel NAV 1.3, and sodium channel NAV 1.6, with a compound at a given dosage.

The compound of the invention, in some embodiments, is a protein, a peptide, a polypeptide, a glycoprotein, or a small molecule. In another embodiment, a compound of the invention is a peptide present in venom.

In one embodiment, the compound of the invention is a peptide comprising or consisting beta-PMTX. In one embodiment, the compound of the invention is a peptide comprising or consisting the amino acid sequence: RIKIGLFQDLSRL (SEQ ID NO: 1). In another embodiment, the compound of the invention is a peptide consisting a derivative or a mimetic of a peptide consisting SEQ ID NO: 1. In another embodiment, a derivative or a peptide of the invention consists at least 4 consecutive amino acids of SEQ ID NO: 1. In another embodiment, a derivative or a peptide of the invention consists at least 5 consecutive amino acids of SEQ ID NO: 1. In another embodiment, a derivative or a peptide of the invention consists at least 6 consecutive amino acids of SEQ ID NO: 1.

In one embodiment, the compound of the invention is a peptide comprising or consisting Conus ermineus venom. In one embodiment, the compound of the invention is a peptide comprising or consisting δ-EVIA. In one embodiment, the compound of the invention is a peptide comprising or consisting the amino acid sequence: DDCIKPIGFCSLPILKNGLCCSGACVGVCADL (SEQ ID NO: 2). In another embodiment, the compound of the invention is a peptide consisting a derivative or a mimetic of a peptide consisting SEQ ID NO: 2. In another embodiment, a derivative or a peptide of the invention consists at least 4 consecutive amino acids of SEQ ID NO: 2. In another embodiment, a derivative or a peptide of the invention consists at least 5 consecutive amino acids of SEQ ID NO: 2. In another embodiment, a derivative or a peptide of the invention consists at least 6 consecutive amino acids of SEQ ID NO: 2.

In one embodiment, the compound of the invention is a peptide comprising or consisting scorpion toxin. In one embodiment, the compound of the invention is a peptide comprising or consisting scorpion css IV (css4) toxin. In one embodiment, the compound of the invention is a peptide comprising or consisting the amino acid sequence: KEGYLVNSYTGCKFECFKLGDNDYCLRECRQQYGKGSGGYCYAFGCWCTHLYEQA VVWPLPNKTCN (SEQ ID NO: 3). In another embodiment, the compound of the invention is a peptide consisting a derivative or a mimetic of a peptide consisting SEQ ID NO: 3. In another embodiment, a derivative or a peptide of the invention consists at least 4 consecutive amino acids of SEQ ID NO: 3. In another embodiment, a derivative or a peptide of the invention consists at least 5 consecutive amino acids of SEQ ID NO: 3. In another embodiment, a derivative or a peptide of the invention consists at least 6 consecutive amino acids of SEQ ID NO: 3.

As used herein, the twenty conventional amino acids and their abbreviations follow conventional usage. Stereoisomers (e.g., D-amino acids) of the twenty conventional amino acids, unnatural amino acids such as alpha-alpha-disubstituted amino acids, N-alkyl amino acids, lactic acid, and other unconventional amino acids may also be suitable components for polypeptides of the present invention. Examples of unconventional amino acids include but are not limited to: 4-hydroxyproline, gamma-carboxy-glutamate, epsilon-N,N,N-trimethyllysine, epsilon-N-acetyllysine, O-phosphoserine, N-acetylserine, N-formylmethionine, 3-methylhistidine, 5-hydroxylysine, .omega.-N-methyllarginine, and other similar amino acids and imino acids (e.g., 4-hydroxyproline). In the polypeptide notation used herein, the lefthand direction is the amino terminal direction and the righthand direction is the carboxy terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

According to some aspects of the invention, the polypeptide of the invention includes homologous polypeptides in which one or more amino acids of SED ID NOs: 1-3 have been added to, deleted from or substituted by conservative substitution of homologous amino acids, on the condition that the resulting modified polypeptide substantially retains the sodium channel selectivity and positive ionotropic effect. “Conservative substitution” refers, in some embodiments, to the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class of residue such as Asn, Gln, or Glu, is a conservative substitution.

According to embodiments of the invention, the resulting modified polypeptides have 80%, 85%, 90%, 95% or more sequence homology with a peptide comprising any one of SED ID NOs: 1-3.

In some embodiments, the invention relates to a DNA molecule encoding a peptide of the invention with or without a leader sequence, a vector comprising the DNA molecule, and a host cell transformed with the vector.

A further aspect of the invention relates to a pharmaceutical composition comprising a polypeptide of the invention, and in particular a pharmaceutical composition for the treatment of heart failure by rendering a positive ionotropic effect on myocytes in general and cardiomyocytes in particular. In another embodiment, the invention relates to a pharmaceutical composition comprising a peptide of the invention in crude venom, and in particular a pharmaceutical composition for the treatment of heart failure by rendering a positive ionotropic effect on myocytes in general and cardiomyocytes in particular.

Recommended dosage of the pharmaceutical composition of the invention may be readily determined by the skilled man of the art. However a rough estimate may be based on a simple consideration related to human envenomation by a venomous scorpion such as the Yellow Israeli Scorpion. An average volume of a single sting of the Yellow Scorpion is about 0.55 μl which contains an average amount of about 57 μg of protein (about 10%), corresponding to about 2 μg of pure toxins (See A. Yahel-Niv and E. Zlotkin (1979) Comparative studies on venom obtained from individual scorpions by natural stings. Toxicon, 17:435-446). The above consideration leads to an approximate dose of 10 μg of the expressed cardiotonic polypeptide per day per adult patient.

In another embodiment, the compound of the invention is provided in an effective amount of 50 mg to 2.5 g. In another embodiment, the compound of the invention is provided in an effective amount of 100 mg to 2 g. In another embodiment, the compound of the invention is provided in an effective amount of 500 mg to 1.5 g. In another embodiment, the compound of the invention is provided in an effective amount of 50 mg to 500 mg.

In another embodiment, a dose comprising a composition of the invention comprises an effective amount of 50 mg to 2.5 g of a compound as described herein. In another embodiment, a dose comprising a composition of the invention comprises an effective amount of 100 mg to 2.0 g of a compound as described herein. In another embodiment, a dose comprising a composition of the invention comprises an effective amount of 500 mg to 1.5 g of a compound as described herein. In another embodiment, a dose comprising a composition of the invention comprises an effective amount of 50 mg to 500 mg of a compound as described herein.

### Treatment

In some embodiments, a compound of the invention is used during a medical procedure wherein cardiac output decreases. In some embodiments, a compound of the invention is used during a medical procedure wherein a risk for decreased cardiac output exists. In some embodiments, a subject of the invention has decreased cardiac output. One skilled in the art, according to the invention, can readily determine that a subject is suffering from a decreased cardiac output. In some embodiments, reduced cardiac output is treated with a compound of the invention regardless of the underlying cause or medical condition.

In some embodiments, a compound of the invention is used to treat heart failure in general. The methods of the invention are effective in treating heart failure which involves reduced systolic function. Thus, in some embodiments, a subject of the invention is afflicted with heart failure. In another embodiment, the subject is afflicted with reduced systolic function. In another embodiment, the subject is afflicted with left ventricular failure (LVF) or congestive cardiac failure (CCF).

A compound of the invention is further used, in some embodiments, for inhibiting or ameliorating a symptom associated with heart failure. A symptom associated with heart failure is a left-sided failure symptom and/or right-sided failure symptom such as: edema, ascites, nocturia, hepatomegaly, jaundice, coagulopathy, dyspnea, orthopnea, fatigability, cardiac asthma, or any combination thereof

In other embodiments, the compound of the invention is used as preventive means to subjects that are susceptible to heart failure. One of ordinary skill in the art can readily identify a subject that is susceptible to heart failure. A subject that can benefit from a treatment with a compound as described herein is afflicted, in some embodiments, with reduced systolic function. A subject that can benefit from a treatment with a compound as described herein is afflicted, in some embodiments, with left ventricular failure (LVF). A subject that can benefit from a treatment with a compound as described herein is afflicted, in some embodiments, with congestive cardiac failure (CCF).

In other embodiments, a compound of the invention is used within a pharmaceutical composition as described herein.

In other embodiments, the compound of the invention is used for treating acute cardiac failure. According to the methods of the invention the composition and the route of administration ensure that the t1/2 of the compound of the invention is few minutes (0.5-30 minutes).

In other embodiments, the compound of the invention is used for treating acute decompensate heart failure (ADHF). According to the methods of the invention the composition and the route of administration ensure that the t1/2 of the compound of the invention is within one hour.

In some embodiments, the compound of the invention is used during resuscitation or cardiopulmonary resuscitation (CPR). The compound of the invention, in some embodiments, is used in advanced life support therapy. The compound of the invention, in some embodiments, is used in pediatric advanced life support (PALS) therapy.

In another embodiment, the compound of the invention is further used for improving organ perfusion. In another embodiment, the compound of the invention is further used for improving organ perfusion during CPR. In another embodiment, the compound of the invention is further used for preventing recurrence of malignant ventricular arrhythmias. In another embodiment, the compound of the invention is further used for increasing conductibility in nodal tissue. In another embodiment, the compound of the invention is further used for protecting the brain from hypoxia.

In another embodiment, the compound as described herein is administered for chronic maintenance of reduced cardiac output. In another embodiment, the compound as described herein is orally administrated. In another embodiment, the compound as described herein is administered via a pump that pumps a composition comprising the compound of the invention according to a desired pharmacodynamic/pharmacokinetic profile set by one of skill in the art.

In another embodiment, the compound as described herein replaces an inotropic or a vasopressor agent. In another embodiment, the compound as described herein is combined with an inotropic and/or a vasopressor agent such as but not limited to: norepinephrine, epinephrine, dopamine, and dobutamine. In another embodiment, the compound as described herein is free of the devastating side effects of alpha and beta adrenergic agents, as well as dopaminergic agents.

In another embodiment, the compound as described herein is utilized as post cardiac surgery therapy. In another embodiment, the compound as described herein is utilized in post-operative cardiac support. In another embodiment, the compound as described herein has unexpected benefits compared to the known agents mentioned such as: no increase in myocardial oxygen consumption, minor or no effects on heart rate, minor or no vasoconstrictive effect, and/or reduced or no tachyphylaxis/waning.

In another embodiment, the compound as described herein is utilized in treating or alleviating symptoms associated with: congestive heart failure secondary to ischemic cardiomyopathy, toxin-induced cardiomyopathy, congenital heart disease, valvular insufficiency, or aortic stenosis. In another embodiment, the compound as described herein is utilized in treating or alleviating symptoms associated with right-sided heart conditions such as PAH, PAH/IPF, and CHD. In another embodiment, the compound as described herein is utilized in treating or alleviating symptoms associated with acute CHF exacerbations or acute myocardial depression (e.g. from sepsis).

The “subject” of the invention, in some embodiment, is a mammal. In one embodiment, the subject is a human. In one embodiment, the subject is an adult. In one embodiment, the subject is an adult afflicted with heart failure. In one embodiment, the subject is a human susceptible to heart failure. In one embodiment, the subject is a farm animal. In one embodiment, the subject is a pet. The phrase “Heart failure” is a condition in which the heart, weakened by disease, fails to pump as well as it should. A subject susceptible to heart failure is a subject afflicted with coronary heart disease. A subject susceptible to heart failure is a subject that is diagnosed with death of heart muscle. In some embodiments, a subject susceptible to heart failure is a subject that experienced a heart attack In some embodiments, a subject susceptible to heart failure is a subject having long-term high blood pressure, uncontrolled diabetes and/or cardiomyopathy. In some embodiments, a compound of the invention can reverse these causes of heart failure. In another embodiment, a subject of the invention is in need of ongoing treatment to relieve the symptoms of heart failure.

In another embodiment, a subject to ne treated by the methods of the invention suffers from the symptoms of heart failure only during physical exertion when the heart cannot cope with the extra pumping activity needed. As heart failure worsens, however, symptoms become more and more debilitating until the subject becomes bedridden, or even immobile. Fortunately, the methods of the invention, can help to control the condition even when it is quite advanced.

### Composition and Delivery

In one embodiment, the compound of the present invention can be provided to the individual per se. In other embodiments, the compound of the present invention is provided to the individual as part of a pharmaceutical composition where it is mixed with a pharmaceutically acceptable carrier.

For treating acute cardiac failure, the composition of the invention, ensures, that compound's t1/2 is between 0.2 to 30 minutes. In another embodiment, for treating acute cardiac failure, the composition of the invention, ensures, that compound's t1/2 is between 0.2 to 15 minutes. In another embodiment, for treating acute cardiac failure, the composition of the invention, ensures, that compound's t1/2 is between 0.2 to 10 minutes. In another embodiment, for treating acute cardiac failure, the composition of the invention, ensures, that compound's t1/2 is between 0.5 to 5 minutes.

For treating acute decompensate heart failure (ADHF), the composition of the invention, ensures, that compound's t1/2 is between 1 to 90 minutes. In another embodiment, for treating ADHF, the composition of the invention, ensures, that compound's t1/2 is between 1 to 75 minutes. In another embodiment, for treating ADHF, the composition of the invention, ensures, that compound's t1/2 is between 1 to 60 minutes. In another embodiment, for treating ADHF, the composition of the invention, ensures, that compound's t1/2 is between 5 to 45 minutes.

A “pharmaceutical composition” refers to a preparation of one or more of the active ingredients described herein with other chemical components such as physiologically suitable carriers and excipients. The purpose of a pharmaceutical composition is to facilitate administration of a compound to a subject. A “compound” of the invention refers to a compound, which is accountable for the biological effect described herein.

In some embodiments, the compound of the invention is a administered with at least one additional compound for treating heart failure or ameliorating symptoms associated with heart failure. “At least one additional compound” is at least one additional compound having an effect on the cardiovascular system. In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In another embodiment, the compound as described herein is administered intravenously. In another embodiment, the compound as described herein is administered via central venous cannula (CVC) or peripheral cannula. In another embodiment, in case the intravenous (IV) access needs to be obtained, a peripheral route, such as the anti-cubital fossa, is usually preferred because it is least invasive, has a lower risk of complications and should not hinder CPR.

In another embodiment, the compound as described herein is administered intraosseously. In another embodiment, intraosseous (TO) access is used if IV access cannot be established.

In another embodiment, the compound as described herein is administered via the tracheal route.

In another embodiment, the compound as described herein is diluted with sterile water or 0.9% normal saline. In another embodiment, the compound as described herein is placed in a pre-filled syringe. In another embodiment, the compound as described herein is administered in combination with any one of: Adrenaline, Amiodarone, Magnesium sulphate, Atropine, Calcium, Sodium bicarbonate, or any combination thereof.

In some embodiments, the compound of the invention is administered with and/or combined with an inotropic and/or a vasopressor agent. In some embodiments, the compound of the invention is administered with and/or combined with an alpha and/or beta adrenergic agent. In some embodiments, the compound of the invention is administered with and/or combined with a dopaminergic agent. In some embodiments, the compound of the invention is administered with and/or combined with norepinephrine, epinephrine, dopamine, dobutamine, or any combination thereof.

In some embodiments, at least one additional compound includes a diuretic. In some embodiments, at least one additional compound includes an ACE inhibitor. In some embodiments, at least one additional compound includes a beta-blocker. In some embodiments, at least one additional compound includes fish oil.

In one embodiment, the phrases “physiologically acceptable carrier” and “pharmaceutically acceptable carrier” which are interchangeably used, refer to a carrier or a diluent that does not cause significant irritation to an organism (subject) and does not abrogate the biological activity and properties of the administered compound. An adjuvant is included under these phrases. In one embodiment, one of the ingredients included in the pharmaceutically acceptable carrier can be for example polyethylene glycol (PEG) or a biocompatible polymer with a wide range of solubility in both organic and aqueous media.

In one embodiment, “excipient” refers to an inert substance added to a pharmaceutical composition to further facilitate administration of an active ingredient. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, Pa., latest edition, which is incorporated herein by reference.

In one embodiment, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In one embodiment, the preparation is administered in a local rather than systemic manner, for example, via injection of the preparation directly into the cardiovascular system. In another embodiment, the preparation is administered directly into the heart.

Oral administration, in one embodiment, comprises a unit dosage form comprising tablets, capsules, lozenges, chewable tablets, suspensions, emulsions and the like. Such unit dosage forms comprise a safe and effective amount of the desired compound, or compounds, as described herein.

In one embodiment, the oral dosage form comprises predefined release profile. In one embodiment, the oral dosage form of the present invention comprises an extended release tablets, capsules, lozenges or chewable tablets. In one embodiment, the oral dosage form of the present invention comprises a slow release tablets, capsules, lozenges or chewable tablets. In one embodiment, the oral dosage form of the present invention comprises an immediate release tablets, capsules, lozenges or chewable tablets. In one embodiment, the oral dosage form is formulated according to the desired release profile of the pharmaceutical active ingredient as known to one skilled in the art.

Peroral compositions, in some embodiments, comprise liquid solutions, emulsions, suspensions, and the like. In some embodiments, pharmaceutically-acceptable carriers suitable for preparation of such compositions are well known in the art. In some embodiments, liquid oral compositions comprise from about 0.012% to about 0.933% of the desired compound or compounds, or in another embodiment, from about 0.033% to about 0.7%

In another embodiment, the pharmaceutical compositions are administered by intravenous, intra-arterial, intra-caediac, or intramuscular injection of a liquid preparation. In some embodiments, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

In one embodiment, injectables, of the invention are formulated in aqueous solutions. In one embodiment, injectables, of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

In another embodiment, the pharmaceutical composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In another embodiment, a compound of the invention or a composition comprising a compound of the invention is delivered from a pump providing a predetermined amount of the compound of the invention over time into the circulation, from a reservoir comprising the compound of the invention. In another embodiment, the reservoir is adapted to stabilize the compound of the invention. In another embodiment, the reservoir is adapted to inhibit the degradation of the compound of the invention. In another embodiment, a compound of the invention or a composition comprising a compound of the invention is attached to or connected to a polymer that has a predetermined release profile. In another embodiment, a compound of the invention or a composition comprising a compound of the invention is attached to or connected to a drug eluting stent. A drug eluting stent is, in some embodiments, is a peripheral or a coronary stent. Drug eluting stents for delivering cardiovascular drugs and/or short peptides are known to one of skill in the art. In another embodiment, the means for administration ensure that the compound of the invention is delivered to a cardiac tissue and not to other tissues including the brain.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Bioactivity Assays

The use of animals in the experiments was approved by the Ethics Committee for Animal Experimentation of The Hebrew University of Jerusalem, and complied with the guidelines of the National Research Council Guide for the Care and Use of Laboratory Animals (NIH Publication no. 85-23, revised 1996).

### Example 1

### The Use of δ-EVIA as a Compound of the Invention Having an Inotropic Activity

δ-EVIA, is a peptide isolated from Conus ermineus venom. The peptide sequence contains 32 amino acid residues and six-Cysteines which forms 3 disulfide bridges (SEQ ID NO: 2). δ-EVIA binds to site 6 of voltage-gated sodium channels and inhibits the transition of the channels to inactivation state, thus increasing the sodium current influx.

The peptide is known in inhibiting sodium channel inactivation in neuronal membranes from amphibians and mammals (Nav1.2a/SCN1A, Nav1.3/SCN3A and Nav1.6/SCN8A) upon binding to receptor site 6, without affecting rat skeletal muscle (Nav1.4/SCN4A) and human cardiac muscle (Nav1.5/SCN5A) sodium channels (Barbier, J., Lamthanh, H., Le Gall, F., Favreau, P., Benoit, E., Chen, H., Gilles, N., Ilan, N., Heinemann, S. H., Gordon, D., Menez, A., and Molgo, J. (2004) A delta-conotoxin from Conus ermineus venom inhibits inactivation in vertebrate neuronal Na+ channels but not in skeletal and cardiac muscles. J Biol Chem 279, 4680-5).

However, to date the activity of δ-EVIA in cardiac tissue was examined.

Expression of Recombinant Delta-Conotoxin EVIA (rδ-EVIA)

The 32 amino acids mature EVIA was expressed as a fusion with a six His-tag and the Trx gene in pET 32a (Novagen). A cleavage site for protease factor Xa was located just before the AVIA gene. Expression was performed in the Origami E. coli strain (Novagen), grown to O.D. 0.6, induced by IPTG (0.2 mM) and incubated overnight at 20° C. Cells were lysed by sonication in Tris-HCl buffer, pH 7.1, centrifuged at 12,000 rpm for 20 minutes at 4° C., mixed with equilibrated nickel-agarose resin (Adar Biotech) for 60 min at 4° C., washed with Tris-HCl buffer and eluted with 250 mM imidazole in Tris-HCl buffer. The imidazole was removed from the elution buffer using 10 KD spin concentration tubes (Milipore). The fusion protein was incubating overnight with protease Factor Xa at 4° C. The mature peptide was purified by filtration through 30 KD spin concentration tubes (Milipore).

### Measurements of Ex-Vivo Heart Mechanical Performance

Animals were euthanized by cervical dislocation following ketamine-xylazine anesthesia (8.5 mg/100 g body wt ketamine in 0.5% xylazine). Hearts were rapidly removed and placed in a physiological solution (containing in mM: 118 NaCl, 24 NaHCO₃, 1.2 KH₂PO₄, 1.2 MgCl₂, 1.2 CaCl₂, 4.2 KCl, and 5.5 glucose at pH 7.4) at 4° C.

The hearts were then mounted on a Langendorff perfusion apparatus and retrogradely perfused via the aorta at a perfusion pressure of 100 cm H₂O. As soon as the perfusion started, a deflated latex balloon (Hugo Sacks Electronics no. 3 or 4) attached to a Statham P23db pressure transducer was inserted into the left ventricle and gradually inflated with saline until maximal systolic pressure at 0-mmHg diastolic pressure was recorded. Left ventricular pressure was recorded using a computerized data-acquisition system (MP100, Biopac Systems, Santa Barbara, Calif.), and rates of pressure development and relaxation were calculated as ±dP/dt/P.

The active substances where infused through the perfusion buffer after steady state was reached, 10-16 min after the onset of perfusion. Data collection was at normothermic temperatures (37° C.).

### δ-EVIA Effects on Ex-Vivo Whole Heart Performances

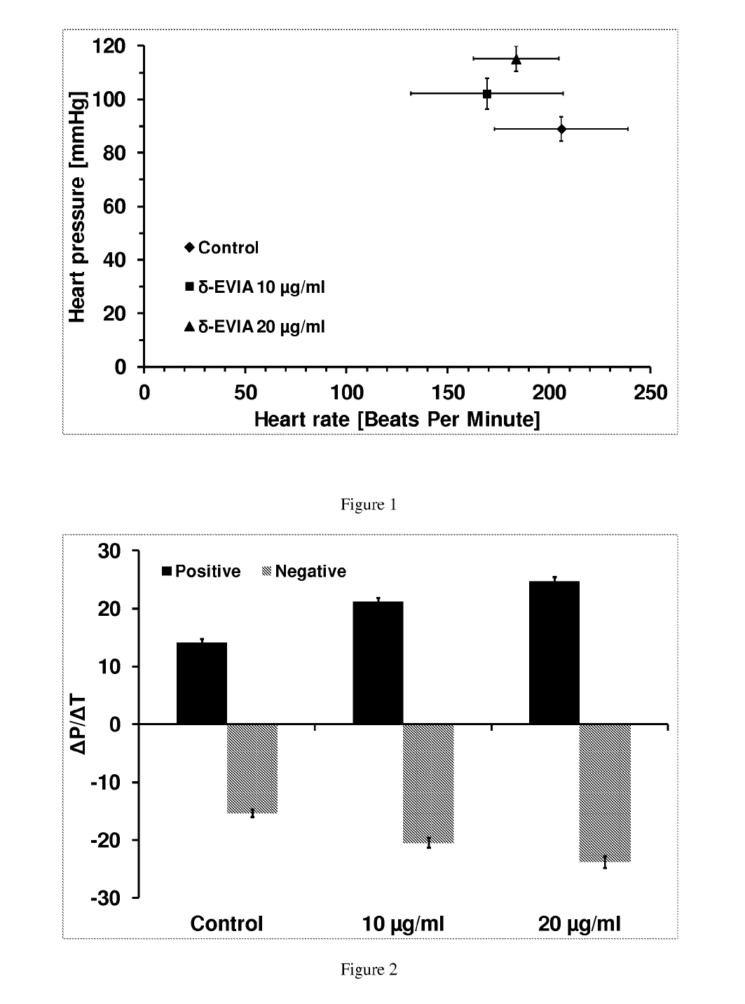

δ-EVIA effects were evaluated on ex-vivo whole heart by using the Langendorff perfusion system, in which the heart is removed from the animal and exposed to the drugs by retrograde perfusion through the aorta. As demonstrate in FIG. 1, δ-EVIA increases heart pressure output and without significantly affecting heart rate.

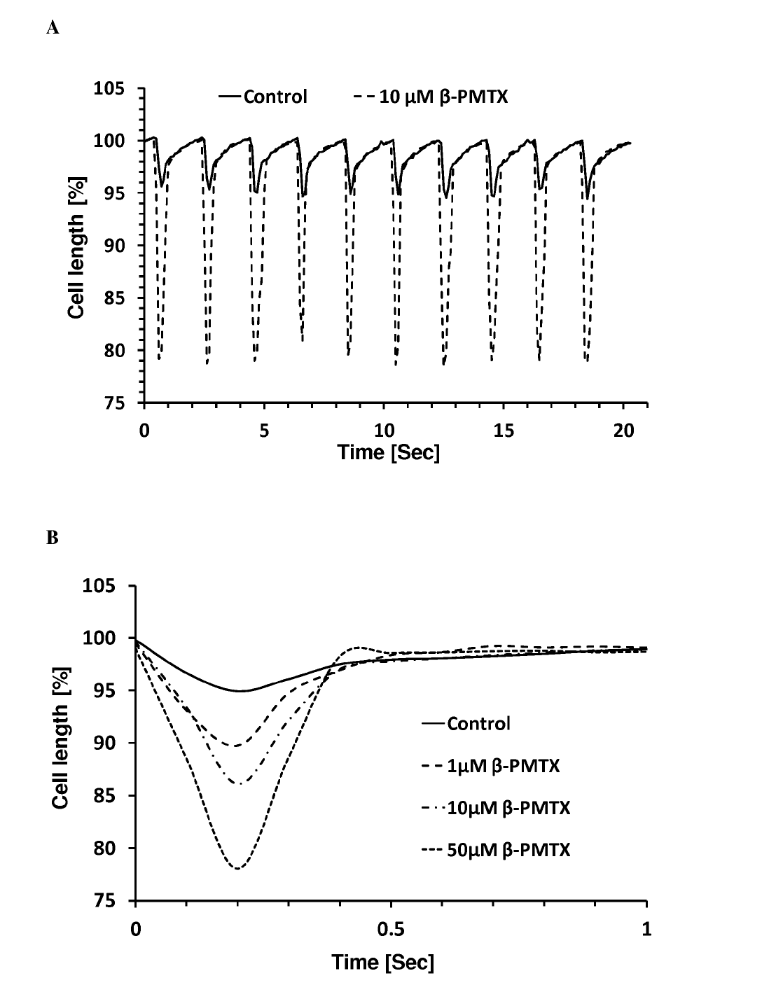

Thus, the δ-EVIA peptide clearly increases the heart ability of producing pressure at a given time (positive ΔP/ΔT) as demonstrate in FIG. 2A and clearly increases the heart ability to regain relaxation (negative ΔP/ΔT).

The currently presented results show that δ-EVIA (a peptide with specific effect on the neuronal Na⁺ channels subtypes but no known effect on skeletal and cardiac muscle Na⁺ channel subtypes), unexpectedly, increased cardiac mechanical performance with no significant effect on its rhythm. There is a long felt need in cardiology for this captured activity. This data provides that compounds modulating the neuronal Na⁺ channel subtypes in the heart muscle are of great value due to their inotropic effect.

### Example 2

### The Use of β-PMTX as a Compound of the Invention Having an Inotropic activity

### Experimental Procedures Solutions

Ca+2-free modified tyrode solution: 120 mM NaCl, 15 mM NaHCO3, 5.4 mM KCl, 5 mM HEPES Na+ salt, 0.25 mM NaH2PO4, 0.5 mM MgCl2, adjust to pH 7.4 with KOH pH 7.4.

Modified tyrode: Added 1 mM Ca+2 to the above buffer.

KB solution: 70 mM KOH, 50 mM glutamic acid, 40 mM KCl, 20 mM taurine, 20 mM KH2PO4, 10 mM glucose, 10 mM HEPES, 0.5 mM EGTA, 3 mM MgCl2, adjust to pH 7.4 with KOH.

Anesthesia-ketamine, 15% xylazin delivered I.P.

Animals used rats weighing 250-280 gr.

### Measurements

All measurements were digitized and recorded on powerLab hardware 16/30 (Adinstruments, Australia). The electrocardiogram (ECG) recorded from subcutaneous leads in Lead II position, amplified on dual bioAmp amplifier (Adinstruments). The blood pressure recorded from the femoral artery that was cannulated with PE 50 polyethylene tube and attached to pressure transducer (Merit, USA). Left ventricular pressure recorded by Millar SPR-121 transducer introduced into the left ventricle via the common carotid artery (Millar instruments, USA) and amplified via PCU-2000 amplifier (Millar). The femoral vain was cannulated with PE-50 for drug and solution administration.

### In-Vivo Protocol

After the animals were anesthetized, cannulated and acquired homodynamic steady state, the animals received 0.5-1 ml worm saline (37° C.) as control injection via the femoral vein as bolus push or via syringe pump (Harvard apparatus 22, USA). Whenever no significant hemodynamic change was followed by the saline injection, the study drug (Beta PMTX or adrenalin) was administered. After regaining base-line, a second dose was administered.

### Isolation of Adult Rat Cardiomyocytes

Adult male Sprague Dawley rats weighing 175-250 g were injected with 0.5 ml of 1000 USP units/ml heparin I.P. 30 minutes prior to anesthetization. The rats were then anesthetized by injecting ketamine/xylazine (8.5 mg/100 g body wt ketamine in 0.5% xylazine) I.P. The heart was removed and was attached to a cannula connected to a series of condensers containing different solutions warmed to 37° C. and bubbled with 95% oxygen/5% CO₂. Then the heart was subjected to reverse Langendorff perfusion through its aorta in a constant perfusion rate of 10 ml/min. While perfusing, a water funnel maintained the temperature of the heart surroundings.

First the heart was perfused with Ca⁺²-containing modified tyrode solution for 2-3 min. Then we the solution was changed to to Ca⁺² free modified tyrode for 5 min. After which, the heart was perfused for 10 min with 100 ml of modified tyrode solution containing 2.5 μM Ca⁺², 17 mg Collagenase type II (Worthington) and 0.8 mg protease type XIV (Sigma). The heart was removed from the cannula and the atria were cut off. The ventricles were soaked into 3 ml KB solution and cut into small pieces and then triturated in a larger volume of KB with a wide bore plastic pipette. The resulted soup like solution was filtered on a silk rug and the cells were refrigerated and stored in the KB solution for up to 24 hours.

### Cardiomyocytes Contraction Measurements

Shortening of rat ventricular cardiomyocytes was measured with an edge-detection video system. Cardiomyocyte shortening was visualized by a Nikon Diaphot 200 inverted microscope attached to a video motion edge detector (Crescent Electronics, Sandy, Utah).

The system integrated by a Computer. Felix software program was used to store and analyze data and for histogram visualization. Experiments were performed at room temperature on intact, rod-shaped cells, which had no spontaneous contractions or microblebs. Cells were placed in a chamber and perfused at 1±0.2 ml/min with Krebs solution containing: 1.2 mM MgSO4, 25 mM NaHCO3, 11 mM Glucose, 4.7 mM KCl, 1.25 mM CaCl2, 1.2 mM KH2PO4 and 118 mM NaCl. pH=7.4 with NaOH.

The cardiomyocytes were field-stimulated (0.5 Hz, square waves), and contractions (amplitude systolic motion) were measured. Recordings are of cell fraction of shortening as a percentage of resting cell length.

### PMTX Effects on Cardiomyocytes Contractions

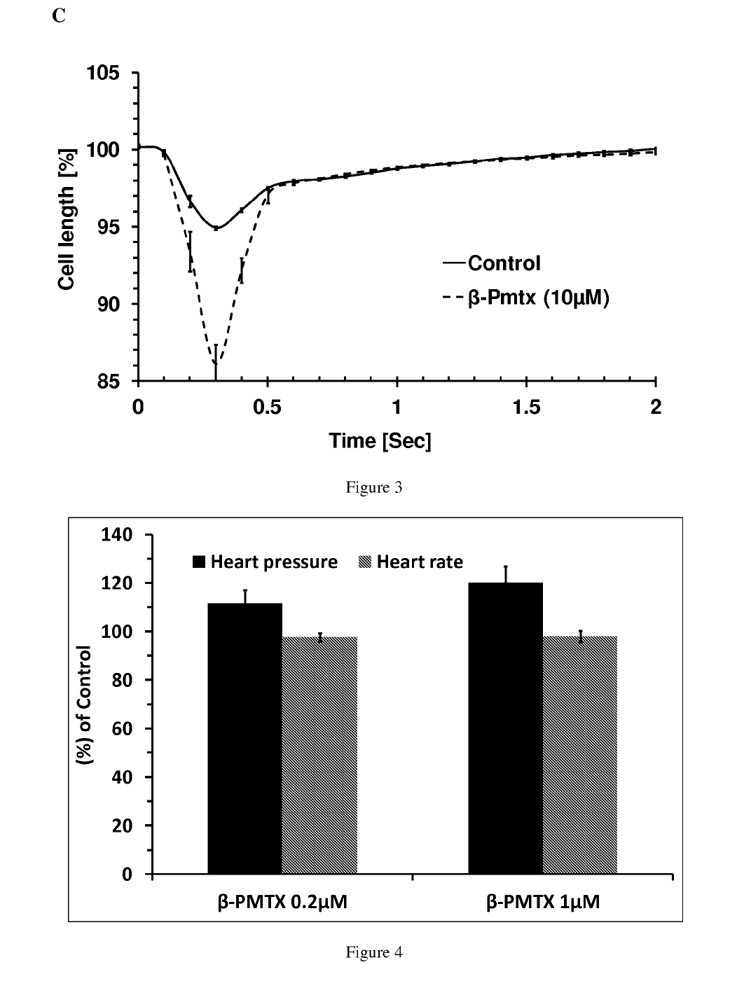

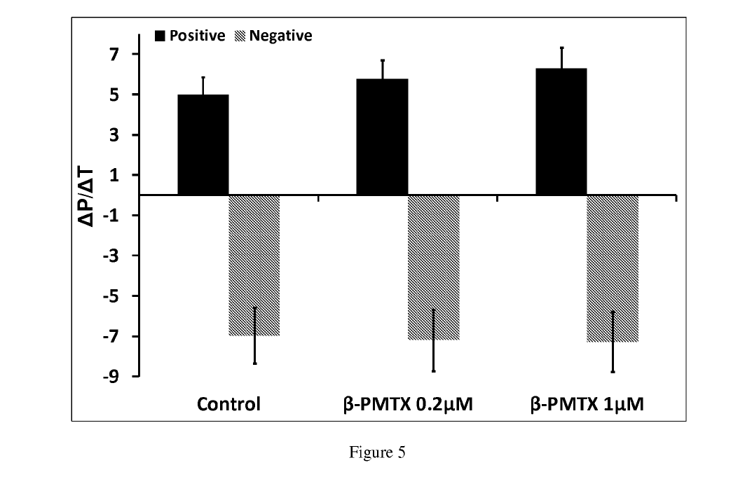

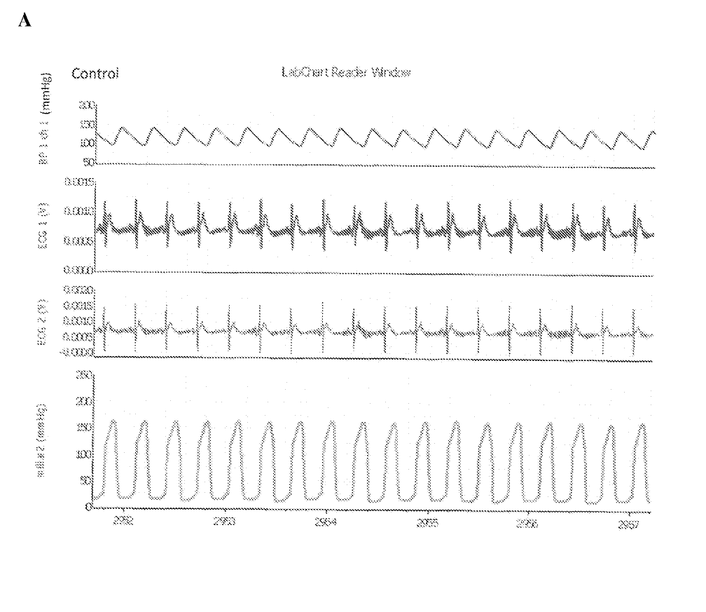

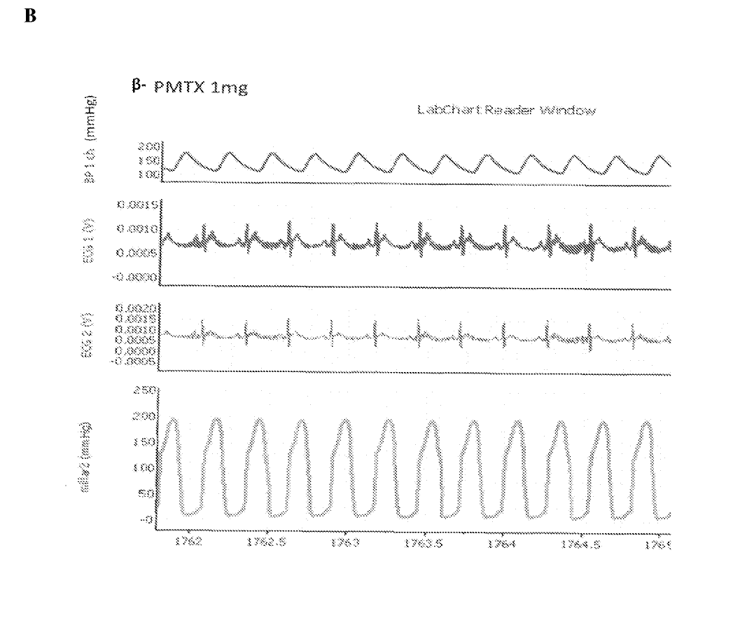

The effects of PMTX on cardiomyocytes contractions amplitude was measured by Video Edge Detection system. The cells were perfused with krebs buffer contain 1.25 mM Ca⁺² and contractions were evoked by external stimulation at 0.5 Hz. PMTX significantly increases the amplitude of cardimyocyte contractions in a concentration dependent manner (FIG. 3C).

Concentration of 10 μM increased the amplitude of cardiomyocyte contractions by 175.5±24.5%, p_{—}6.8 E-06. This effect was not accompanied by change in twitch duration, configuration and in its time to peak. At this range of toxin concentrations no spontaneous twitches were observed.

### The In-Vivo Effect of PMTX on the Cardiac Performance of Mammals.

In order to verify the effect of β-PMTX on the cardiac performance of mammals in-vivo, β-PMTX and adrenalin were injected to live anesthetized rats cannulated with blood pressure (BP), Left ventricular pressure (LvP) and ECG measurement devices.

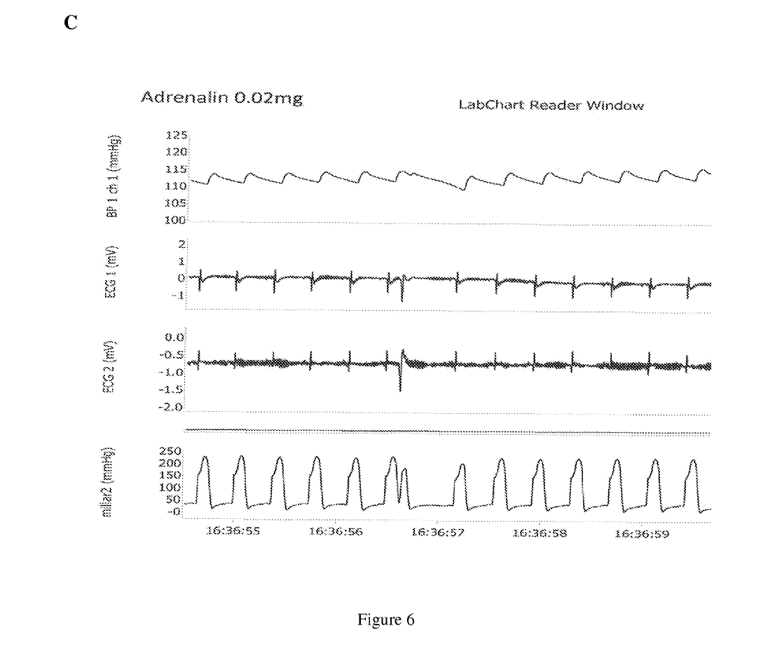

β-PMTX increased LvP from 100 mmHg in the control to 150 mmHg (dose of 0.5 mg) and to 200 mmHg (dose of 1 mg) and in consequence the arterial blood pressure (ABP) in concentration dependent manner with no induction of rhythm disturbances in dose range between approximately 0.5 mg to 2 mg (≈concentration range of 50 μg/ml-250 μg/ml) (see FIG. 6).

Adrenalin unlike β-PMTX, although increased heart contractility from 100 mmHg in the control to 200 mmHg (dose of 0.1 mg) and ABP, also induced rhythm disturbances in low concentrations.

These in-vivo results further confirm the previous in-vitro findings obtained for isolated cardiomyocytes and in ex-vivo heart (removed heart) in which β-PMTX increased LvP and consequence the BP with no induction of rhythm disturbances.

1-29. (canceled)

30. A method for increasing cardiac output in a subject in need thereof, comprising the step of administering to said subject a composition comprising a compound which activates at least one of the sodium channels: NAV 1.1, NAV 1.3, NAV 1.6 or a combination of two or more of such sodium channels, thereby increasing cardiac output in a subject in need thereof.

31. The method of claim 30, wherein said compound does not activate the sodium channel NAV 1.5; or said compound activates sodium channel NAV 1.5 to a lesser extent compared to said compound activation of said sodium channel NAV 1.1, said sodium channel NAV 1.3, and said sodium channel NAV 1.6.

32. The method of claim 30, wherein said compound is a protein or a small molecule.

33. The method of claim 30, wherein said compound comprises a protein selected from the group comprising: a peptide comprising the amino acid sequence of SEQ ID NO: 1, a peptide comprising the amino acid sequence of SEQ ID NO: 2, a peptide comprising the amino acid sequence of SEQ ID NO: 3, a beta-scorpion toxin, or any combination thereof.

34. The method of claim 30, wherein said compound consists the amino acid sequence of: SEQ ID NO: 1, SEQ ID NO: 2, or SEQ ID NO: 3.

35. The method of claim 30, wherein said increasing cardiac output is increasing the: amplitude of a cardiomyocyte contraction, force or energy of cardiac contraction, left ventricular pressure, or any combination thereof.

36. The method of claim 30, wherein said increasing cardiac output is substantially free of: inducing a cardiac rhythm disturbance, affecting an electrical property of the heart, or both.

37. The method of claim 30, wherein said increasing cardiac output is treating heart failure.

38. The method of claim 30, wherein said increasing cardiac output is reducing systolic function.

39. The method of claim 30, wherein said increasing cardiac output is treating left ventricular failure (LVF).

40. The method of claim 30, wherein said increasing cardiac output is treating congestive cardiac failure (CCF).

41. The method of claim 30, wherein said increasing cardiac output is inhibiting or ameliorating a symptom associated with heart failure.

42. The method of claim 41, wherein said symptom is: edema, ascites, nocturia, hepatomegaly, jaundice, coagulopathy, dyspnea, orthopnea, fatigability, cardiac asthma, or any combination thereof.

43. A method for increasing cardiac output in a subject in need thereof, comprising the step of administering to said subject a composition comprising a peptide, said peptide comprising the amino acid sequence of SEQ ID NO: 1, SEQ ID NO: 2, SEQ ID NO: 3, or any combination thereof, thereby increasing cardiac output in a subject in need thereof.

44. The method of claim 43, wherein said peptide is 50 mg to 2.5 g of said peptide.

45. The method of claim 43, wherein said increasing cardiac output is substantially free of: inducing a cardiac rhythm disturbance, affecting an electrical property of the heart, or both.

46. The method of claim 43, wherein said increasing cardiac output is: treating heart failure, reducing systolic function, treating left ventricular failure (LVF), treating congestive cardiac failure (CCF), or any combination thereof.

47. The method of claim 43, wherein said increasing cardiac output is inhibiting or ameliorating a symptom associated with heart failure.

48. The method of claim 47, wherein said symptom is: edema, ascites, nocturia, hepatomegaly, jaundice, coagulopathy, dyspnea, orthopnea, fatigability, cardiac asthma, or any combination thereof.


SEQUENCE LISTING (3 sequences)

SEQ ID NO: 1 - protein, 13 aa, Spider wasp
RIKIGLFQDLSRL

SEQ ID NO: 2 - protein, 32 aa, Conus ermineus
DDCIKPIGFCSLPILKNGLCCSGACVGVCADL

SEQ ID NO: 3 - protein, 66 aa, scorpion
KEGYLVNSYTGCKFECFKLGDNDYCLRECRQQYGKGSGGYCYAFGCWCTHLYEQAVVWPLPNKTCN